(19) United States
(12) Patent Application Publication
(10) Pub. No.: US 2008/0008676 A1
(43) Pub. Date: Jan. 10, 2008

(54) Deodorant composition comprising metallic deodorizing agent

(75) Inventors: Radhakrishnan Janardanan Nair, Kobe (JP); Yoko Matsuki, Ashiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/825,430

(22) Filed: Jul. 6, 2007

Related U.S. Application Data
(60) Provisional application No. 60/818,924, filed on Jul. 6, 2006.

(51) Int. Cl. A61L 9/012; A61K 33/26; A61K 33/30; A61K 33/34; A61K 33/38

(52) U.S. Cl. 424/76.4; 424/618; 424/638; 424/641; 424/646

(57) ABSTRACT

A deodorant composition comprising (i) a metallic deodorizing agent selected from copper, zinc, silver, platinum, nickel, iron, cobalt, a copper compound, a zinc compound, a silver compound, a platinum compound, a nickel compound, an iron compound, a cobalt compound and a mixture thereof, (ii) an carboxylic group-containing gelling polymer; and a liquid media. The deodorant composition has a three-dimensional shape selected from a block shape, a sphere shape, an ellipsoid shape, a cone shape and a cylinder shape and has a surface area of from about 24 mm² to about 24 mm².

### CROSS REFERENCE TO RELATED APPLICATION

This application claims the benefit of U.S. Provisional Application No. 60/818,924, filed Jul. 6, 2006.

### FIELD OF THE INVENTION

The present invention relates to a deodorant composition suitable for deodorizing malodors in the living environment. In particular, the present invention relates to deodorant composition comprising a carboxylic group-containing gelling polymer and a metallic deodorizing agent.

### BACKGROUND OF THE INVENTION

There are a variety of chemical compounds in the living environment giving out unpleasant smell to the surroundings or even being harmful to the health of human beings. For example, ammonia, amines, hydrogen sulfide, mercaptans, lower fatty acids, aldehydes, etc. are typical chemical compounds responsible for the malodors in kitchens, toilet room, basements, cars, etc. Many types of deodorizers have been developed to deodorize or absorb these malodors. Deodorizers available in the market typically mask malodors by emitting fragrances, or physically absorb and/or chemically react with the malodorous substances. Materials usually used as an absorbent include activated carbon, zeolites, cyclodextrins, bentonites, etc. Materials usually used to react with malodorous substances include chlorine dioxide, hypo chloride, ozone, polymers comprising functional groups, etc. These materials decompose malodorous compounds by oxidization, reduction or neutralization reactions.

Metal or metal compounds have been suggested to be used in deodorizing products. Normally, a deodorant product is provided and used in a package comprising a plurality of individual bodies of a deodorant composition comprising deodorant active agents. Inventors of the present invention have found that for such a deodorant product, besides the content level of a metal or metal compound contained in the deodorant composition, the shape and surface area of the individual body of the deodorant composition also affect the deodorant performance of the deodorant product. Thus, the need for an optimal shape and surface area of a deodorant composition still exists. In addition, for a gel deodorant composition comprising a metal or metal compound, a formulator may find himself in a dilemma with regard to formulating the metal or metal compound into the gel deodorant composition as the level of a metal or metal compound normally needed in a deodorant composition will give the gel an unpleasant color and/or poor color integrity. For example, when a copper compound is formulated into a gel deodorant composition at a high level, the gel may have a dark blue color which is not appealing to consumers. Likewise, when a nickel compound, an iron compound, a cobalt compound or a platinum compound is formulated into a gel deodorant composition at a high level, the color of the gel may become dark and when a zinc oxide is used in a gel at a high level, the gel may have a whitish color and become undesirable opaque. A silver compound, on the other hand, may change its color when included at a high level in a gel deodorant composition and upon contact with air in use. Thus, there is still a need for a metal or metal compound-containing gel deodorant composition which is effective in deodorizing malodors in living environment, especially toilet room and meanwhile has an appealing color and improved color integrity.

### SUMMARY OF THE INVENTION

The present invention related to a deodorant composition comprising a carboxylic group-containing gelling polymer and from about 0.005% to about 2% by weight of a metallic deodorizing agent selected from copper, zinc, silver, platinum, nickel, iron, cobalt, a copper compound, a zinc compound, a silver compound, a platinum compound, a nickel compound, an iron compound, a cobalt compound and a mixture thereof, wherein said deodorant composition has a three-dimensional shape selected from a block shape, a sphere shape, an ellipsoid shape, a cone shape and a cylinder shape and having a surface area of from about 24 mm² to about 2400 mm². It has been surprisingly found that a deodorant product comprising a plurality of individual bodies of the deodorant composition herein is effective in deodorizing malodors in the living environment, especially a toilet room and has an appealing color and improved color integrity.

As mentioned above, when a metal or metal compound is formulated into a gel deodorant composition, typically, the level necessary to give a satisfied deodorant performance will make the gel deodorant composition have an unpleasant color and/or poor color integrity. Extensive research has been done to address this problem, and it has been found now that a gel deodorant composition in a three-dimensional shape selected from a block shape, a sphere shape, an ellipsoid shape, a cone shape and a cylinder shape having a surface area of from about 24 mm² to about 2400 mm² can provide satisfactory deodorant performance with a relatively low level of a metal or metal compound, and therefore, the problems associated with the high content level of a metal or metal compound in the gel deodorant composition, such as the unappealing color and poor color integrity are solved. Without intending to be bound by theory, it is believed that a three-dimensional shape selected from a block shape, a sphere shape, an ellipsoid shape a cone shape and a cylinder shape having a surface area of from about 24 mm² to about 2400 mm², or from about 150 mm² to about 600 mm² increase the contact of the metal or metal compound with the malodorous compounds. Furthermore, it is also believed that in a deodorant product, individual bodies of the deodorant composition having the three-dimensional shape in the above size range do not pack to each other closely and the space in-between the individual bodies facilitates the flow of air. As a result, a gel deodorant product having an appealing color, an improved color integrity and consumer-noticeable deodorizing performance is provided.

In another aspect, the present invention relates to a deodorant product comprising a plurality of the individual bodies of the deodorant composition herein.

In another aspect, the present invention relates to a method of making a gel deodorant composition having a desired three-dimensional shape.

In still another aspect, the present invention relates to a method of deodorizing a toilet.

In yet another aspect, the present invention relates to use of a deodorant composition for the manufacture of a deodorizer for deodorizing a toilet.

### DETAILED DESCRIPTION OF THE INVENTION

All percentages, ratios and proportions herein are by weight of the composition, unless otherwise specified. All temperatures are in degrees Celsius (° C.) unless otherwise specified.

As used herein, the term “comprising” and its derivatives means are intended to be open ended terms that specify the presence of the stated features, elements, components, groups, integers, and/or steps, but do not exclude the presence of other, unstated features, elements, components, groups, integers, and/or steps.

As used herein, the term “deodorant product” means a deodorant product for sale and use. The deodorant product comprises a plurality of individual bodies of the deodorant composition of the present invention packed together in a package.

As used herein, the term “amine type malodors” means malodors which are caused by a compound comprising an amine group. Typical compounds in the living environment giving out amine type malodors include, but are not limited to ammonia, trimethyl amine, triethylamine, etc.

As used herein, the term “acid type malodors” means malodors which are caused by a compound comprising a carboxylic acid group. Typical compounds in the living environment giving out acid type malodors include, but are not limited to isovaric acid, butyric acid, propionic acid, etc.

As used herein, the term “sulfur type malodors” means malodors which are caused by a compound comprising sulfur. Typical compounds in the living environment giving out sulfur type malodors include, but are not limited to hydrogen sulfide, methylmercpatan, methyl sulfide, dimethyl sulfide, etc.

As used herein, the term “average particle diameter” means the average particle diameter measured along the longest axis of a given particle material as determined by conventional analytical techniques such as, microscopic determination utilizing a scanning electron microscope (SEM).

As used herein, the term “surface area” means the summation of the areas of exposed surfaces of a three-dimensional shape. For example, when the three-dimensional shape is a cuboid having a length, breadth and a height of a, b and c, then, the surface area of the cuboid is 2(ab+ac+bc), when the three-dimensional shape is a sphere shape having a radius of r, the surface area of the sphere is 4 πr², etc. For irregular shapes, there are several methods which can be employed for measuring the surface area described in the literature. For example, three dimensional digital imaging by digital optical microscopic is one of the methods widely used for measuring surface area of irregular shaped three dimensional structures with great accuracy.

The deodorant composition herein comprises a metallic deodorizing agent, a carboxylic group-containing gelling polymer and a liquid media. The deodorant composition herein may further comprise a deodorant polymer, a preservative, a solubilizer, a UV absorbent, a perfume, a dye and a mixture thereof.

### Metallic Deodorizing Agent

The deodorant composition herein comprises from about 0.005% to about 2%, or from about 0.01% to about 1%, or from about 0.05% to about 0.5% by weight of a metallic deodorizing agent selected from copper, zinc, silver, platinum, nickel, iron, cobalt, a copper compound, a zinc compound, a silver compound, a platinum compound, a nickel compound, an iron compound, a cobalt compound and a mixture thereof. Metal or metal compound is known to be effective in deodorizing sulfur type malodors which are common in living environment, especially toilet room, by reacting with the sulfur-containing chemicals. However, as discussed hereinabove, while it is desirable to formulate a high level of metal or metal compound in a deodorant composition to improve the deodorant performance, where the level of a metal compound in a gel deodorant composition is higher than 2%, the gel deodorant composition will have an unappealing color, such as dark blue and also a poor color integrity. On the other hand, where the level of a metal or metal compound in the gel deodorant composition is lower than 0.005%, the deodorant composition will not give satisfactory deodorant performance.

Metal compound useful herein can be a metal oxide or an organic or inorganic salt of a metal. Where a metal or water-insoluble metal compound is used, a water-insoluble oxides or salts of a metal, the metal or water-insoluble metal compound are preferably added into the deodorant composition in a fine powder form having an average particle diameter of from about 20 nm to about 20 microns. Suitable water-insoluble metal compound useful herein include copper monoxide, copper suboxide, zinc oxide, nickel oxide, etc.

Suitable metal salt useful herein include chloride, sulfate and carbonate of copper, zinc, silver, platinum, nickel, iron and cobalt, as well as a complex of copper, zinc, platinum, silver, iron, cobalt with carboxylic acids or carboxylic acid-containing polymers.

Copper chloride, copper sulfate, copper carbonate, zinc chloride, zinc sulfate, silver and a mixture thereof are the most preferred metallic deodorizing agent useful herein.

### Carboxylic Group-Containing Gelling Polymer

As used herein, “carboxylic group-containing gelling polymer” means a polymer comprising a unit of carboxylate or carboxylic acid and the polymer swells upon absorbing a liquid media, such as water and/or other solvent and forms a gel. Gel is typically considered to be a colloid in which the dispersed phase has combined with the dispersion medium to produce a semisolid material, such as a jelly. As a consumer preferred deodorant product form, gel is used as a carrier of the other components of the deodorant product. Furthermore, the carboxylic group-containing gelling polymer herein may also play the function of a deodorant active agent since the carboxylic group contained therein reacts with the amine groups in a chemical compounds responsible for the amine type malodors.

Suitable carboxylic group-containing gelling polymers herein include homepolymers and copolymers comprising a monomer of olefinically unsaturated carboxylic acid or anhydrides that contain at least one carbon-to-carbon olefinic double bond. Examples of such monomers include acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-sterylacrylic acid, itaconic acid, citroconic acid, mesaconic acid, glutaconic acid, aconitic acid, maleic acid, fumaric acid, tricarboxyethylene, maleic acid anhydride, alkali metal salt of acrylic acid or methacrylic acid, butyl(meth)acrylate, ethyl(meth)acrylate, methyl(meth)acrylate, isobutyl(meth)acrylate, 2-ethylhexyl(meth)acrylate, and a mixture thereof. The carboxylic group-containing gelling polymer can be a cross-linked or non cross-linked polymer. There are no specific limits on the way of cross-linking the polymers or the cross-linking agent can be used. Examples of cross-linking agent useful herein include tetraallyl ethoxyethane, 1,1,1-trimethylopropanetricrylate, methylene bisacrylamide, diethylene glycaol diacrylate, triallylamine, allyl methacrylate, tetraallyloxyethane. Typically, the cross-linking agent is used in amounts ranging from 0.0005 to 5 parts by weight per 100 parts by weight of monomers (including any possible co-monomers) used. These polymers can be used either solely or in the form of a mixture of two or more different polymers. Examples of these polymer materials are disclosed in U.S. Pat. No. 3,661,875, U.S. Pat. No. 4,076,663, U.S. Pat. No. 4,093,776, U.S. Pat. No. 4,666,983, and U.S. Pat. No. 4,734,478.

Preferably, the carboxylic group-containing gelling polymers is a partially neutralized cross-linked polyacrylic acid, such as poly (sodium acrylate/acrylic acid) and cross-linked isobutylene-maleic acid anhydride copolymer. Commercially available gelling polymers herein include Aqualic™ from Nihon Shokubai (Tokyo, Japan), Aquakeep™ from Sumitomo Seika (Osaka, Japan), Aquabeads™ from Kuraray (Osaka, Japan) and Hysorb™ from BASF (Germany). More examples of carboxylic group-containing gelling polymers can be found in the book entitled Modem Superabsorbent Polymer Technology, Ed by Fredric L. Bucholz and Andrew T. Graham, published by John Wiley & Sons, Inc., 1998.

The deodorant composition herein comprises from about 0.5% to about 40%, or from about 1% to about 20%, or from about 4% to about 10% by weight of a carboxylic group-containing gelling polymer.

### Liquid Media

The liquid media useful herein is typically selected from water, oil, an organic solvent, such as an alcohol, and a mixture thereof. In an embodiment herein, the liquid media is water.

Typically, the deodorant composition herein comprises from about 50% to about 95%, or from about 80% to about 90% by weight of a liquid media.

### Deodorant Polymer

According to one embodiment, the deodorant composition herein further comprises a deodorant polymer comprising both “cationically dissociating groups” and “anionically dissociating groups”.

The term “cationically dissociating groups” as used herein means those ion-exchange groups whose counter ion is a cation. A typical cationically dissociating group is an acid group. Cationically dissociating groups have the ability to adsorb polar substances and are capable of releasing a proton (hydrogen ion) to enter into neutralizing reaction with basic substances, such as ammonia or amines. As a result, the basic substances can be removed. One or more cationically dissociating groups may be introduced into the polymer. Examples of such cationically dissociating groups include a carboxyl group, a sulfate group, a phosphate group, a sulfoethyl group, a phosphomethyl group and a carbomethyl group, etc. Preferred cationically dissociating groups include a sulfate group and a carboxyl group. Monomers that have such cationically dissociating groups and that are useful herein include, for example, acrylic acid, methacrylic acid, allylsulfonic acid, vinylsulfonic acid, styrenesulfonic acid and salts thereof, and 2-acrylamido-2-methylpropanesulfonic acid.

The term “anionically dissociating groups” as used herein means those ion-exchange groups whose counter ion is an anion. Anionically dissociating groups have the ability to absorb polar substances and are capable of entering into neutralizing reaction with acidic substances, such as, hydrogen sulfide or mercaptans. As a result, the acidic substances can be removed. One or more kinds of anionically dissociating groups may be introduced into the polymer. Examples of anionically dissociating groups include quaternary ammonium group and primary, secondary, and tertiary amino or amido groups, such as amino group, methylamino group, dimethylamino group, and diethylamino group. Monomers having such anionically dissociating groups and that are useful in the present invention include, for example, vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, acrylamide, allylamine, N,N-dimethylacrylamide, N,N-dimethylaminoethylacrylate and N,N-dimethylaminopropyl acrylamide.

Specifically preferred deodorant polymers are those comprising both carboxyl group and amino group, such as copolymers of acrylic acid with diethylamino ethyl methacrylate, dimethylamino ethyl acrylate, dimethylamino ethyl methacrylate, diethylamino ethyl acrylate, diethylamino methyl methacrylate, tert-butylamino ethyl acrylate, tert-butylamino ethyl methacrylate, dimethylaminopropyl acrylamide, acrylamide, allylamine, N,N-dimethylacrylamide, N,N-dimethylaminoethyl acrylate, or N,N-dimethylaminopropyl acrylamide.

According to another embodiment herein, the deodorant polymer is grafted onto a substrate. By grafting, it means that the polymer is bonded to the substrate at specific sites. Substrates useful herein are not limited in any particular way and can be any materials able to link with the polymer. Illustrative substrates useful for grafting the deodorant polymers include aldohexose, glucose, starch and cellulose. Without intending to be bound by theory, it is believed that the substrate herein is used to support the deodorant polymers containing functional groups. By grafting the deodorant polymers onto the substrate, the contact of the functional groups in the deodorant polymers with the chemical compounds to be absorbed is increased, and thus a better deodorizing performance is obtained.

The deodorant polymer can be grafted onto the substrate by any known polymerization method, such as a reaction initiator polymerization method, a thermal polymerization method, or an ionizing radiation polymerization method. According to one embodiment herein, the graft deodorant polymer is obtained by mixing the monomers and substrate to give a mixture which is then subjected to γ-ray radiation, as described in Japanese Patent No. Publication Kokai No. 2003-887551.

The deodorant composition herein comprises from about 1% to about 40%, or from about 3% to about 15%, or from about 5% to about 10% by weight of a deodorant polymer, if present.

### Other Ingredients

The deodorant composition herein may further comprise adjunct ingredients selected among a perfume, a dye, a stabilizer, a UV absorbent, a preservative and a mixture thereof.

A perfume may further added into the deodorant composition herein at an amount of from about 0.1% to about 5% by weight.

A highly preferred ingredient in the present invention is a UV protector which is used herein to describe a material which absorbs, blocks and/or reflects UV light so as to reduce UV damage. Specifically, polymer molecules in the deodorant composition may degrade and/or break when exposed to light energy. Many light wavelengths, especially in the UV spectrum are known to affect polymer molecules by breaking and/or weakening the internal chemical bonds between monomers. This may cause the shape of the gel deodorant composition herein to become deformed or make the gel appear to be melting over time. In an extreme case, the shape may be destroyed if excessive breaking of molecules occurs because of exposure to light during manufacture, shipping, storage, and/or use.

Useful UV protectors include the UV absorber SEESORB™ 101, available from Shipro Kasei Kaisha (Osaka, Japan), which can be absorbed or otherwise incorporated into the gel. SEESORB™ 101 is a benzophenone based UV absorber. Also useful herein are benzo triazole based UV absorbers such as SEESORB™ 701, also available from Shipro.

Other examples of UV protectors which can be used alone or as a mixture with another UV protectors or with an anti-oxidant include the CYASORB™ UV series from American Cyanamid Co. (Wayne, N.J., USA) and the Tinogard™ TL series from Ciba Specialty Cehmicals Co. (Basel, Switzerland). Such UV protectors may be incorporated into any relevant portion of the product, for example, in to the packaging, into or onto the gel, etc.

Preservatives known in the art may also be useful herein. An example of a preservative is phenoxyethanol.

Anti-oxidants known in the art may also be useful herein to prevent degradation and/or damage to the gelling polymer, perfume, and/or other ingredients in the deodorant composition. While such anti-oxidants are well-known in the art, an example of a preferred anti-oxidant is SEENOX-BCS™ available from Shipro.

### Three-Dimensional Shape

The deodorant composition herein has a three-dimensional shape selected from a block shape, a sphere shape, an ellipsoid shape, a cone shape and a cylinder shape, the three-dimensional shape has a surface area of from about 24 mm² to about 2400 mm², or from about 150 mm² to about 600 mm². As used herein, “block shape” means a polyhedron with no curved surfaces or edges. All sides of a block shape are polygons and all edges of a block shape are line segments. Notwithstanding the above definition, it should be understood that polyhedrons having slightly curved surfaces or edges due to the actual manufacturing capability are not intended to be excluded from the range of block shape herein. Preferably, the deodorant composition herein has a block shape selected from the group consisting of a cube, a cuboid, a parallelepiped and an oblique rectangular prism. By “oblique rectangular prism”, it means a voluminous body having six sides, wherein three pairs of parallel and equally shaped and sized sides exist and wherein one pair of sides are in the shape of a parallelogram and the remaining two pairs of sides are of rectangular shape.

The three-dimensional shape described hereinabove is preferred since a deodorant product for sale and use comprises a plurality of individual bodies of the deodorant composition packed in a package, spaces in-between the individual bodies of the deodorant composition herein facilitate flow of air and allow the air to penetrate into such spaces, thus, contact of the deodorant actives with the malodorous compounds to be removed from the environment is increased. Without intending to be bound by theory, it is believed that where the three-dimensional shape has a surface area of lower than 24 mm², the individual gel body of the deodorant composition tends to be packed closely in a deodorant product because the gel is soft and in a small size. As a result, flow of air in-between the individual gel bodies is blocked. On the other hand, where the three-dimensional shape has a surface area of larger than 2400 mm², the total surface area of the deodorant composition per unit amount is decreased due to the large size of a single body. As a result, contact of malodorous compound with the metallic deodorizing agent in a deodorant product is limited.

### Process of Making a Gel Deodorant Composition in the Three-Dimensional Shape

The gel deodorant composition in the three-dimensional shape herein can be prepared by any known process, such as by mixing all the ingredients and forming a gel deodorant composition, then cutting the gel into a desired shape and size. Preferably, the gel deodorant composition having the three-dimensional shape and surface area described herein is prepared by using a gelling polymer having a preformed three-dimensional shape selected from a block shape, a sphere shape, an ellipsoid shape, a cone shape and a cylinder shape. Specifically, this preferred process comprises steps of preparing a gelling polymer having a preformed 3-dimensional shape; premixing and homogenizing other ingredients of the deodorant composition in a mixer to form a premix; and adding the premix into the gelling polymer and allowing the gelling polymer to adsorb the premix to form a gel deodorant composition, wherein the preformed shape of the gelling polymer is selected from a block shape, a sphere shape, an ellipsoid shape, a cone shape and a cylinder shape, and wherein the weight ratio of the gelling polymer to the premix is from about 1:200 to 1:1, or from about 1:30 to about 1:10. One of the methods for preparing a gelling polymer having a desired three-dimensional shape is suspension polymerization which is described in detail in the book entitled Modem Superabsorbent Polymer Technology, Ed by Fredric L. Bucholz and Andrew T. Graham, published by John Wiley & Sons, Inc., 1998. Gelling polymers having a preformed sphere shape are commercially available as Aquabeads™ from Kuraray (Osaka, Japan).

### Deodorant Product

A plurality of individual bodies of the deodorant composition herein is packed together as a deodorant product for sale and use. Preferably, the deodorant product is provided in a sealed package to prevent the loss of moisture and thus the deformation of the three-dimensional shape of the individual bodies during shipment and storage. Packaging materials providing such function are known in the art.

### Test Methods

### Preparation of an Air Stock with Desired Concentration of Malodorous Compounds

An air stock with desired concentration of a malodorous compound can be prepared by the following method.

A single cock 25 liter Tedlar™ bag (Shibao Shoten, Osaka, Japan) fitted with an open/close valve through which gas can be injected and released is filled with about 20 to 25 liters of clean air using an air-pump.

Standard malodorous compound, such as hydrogen sulfide or ammonia is fed into the Tedlar bag through the valve. After adding a small volume of a malodorous compound, check the concentration of the compound with Gastec's standard detector tube system comprising a gas tube (Model 4H, 4HM, 4M, 4L, 4LL, 4LK, 4LB, 4LT for hydrogen sulfide and Model 3HM, 3M, 3LA and 3L for ammonia, the appropriate model is selected according to the target concentration of the compound and the instruction in the Gastec Handbook: Environmental Analysis Technology published by Gastec Corporation, March 2003, 4^{th} Edition.) and a gas sampling pump (GV-100 gas sampling pump), supplied by Gastec Corporation (Kanagawa, Japan). Adjust the concentration of the malodorous compound to an appropriate level using the Gastec detector tube, for the bag containing hydrogen sulfide, adjust the concentration of hydrogen sulfide to be 100 ppm, and for the bag containing ammonia, adjust the concentration of ammonia to be 200 ppm. After that, shake the bag well and leave it for 5 minutes and detect the concentration again using the detector to confirm the desired concentration.

### Measuring the Deodorant Performance of the Deodorant Composition

Deodorant performance of the deodorant composition herein against a malodorous compound is measured as follows.

A 5 liter Tedlar™ bag is prepared with one corner of the bag being cut. 3.0 grams of a deodorant composition is prepared and put on a small glass Petri dish which is then placed inside the Tedlar bag. After completely pressing the air inside the Tedlar bag out, the cut-opened corner of the bag is sealed completely without any substantial leakage using a heat sealer.

Fill the 5 liter Tedlar™ bag having the deodorant composition sample inside with the air stock comprising a predetermined concentration of a malodorous compound through a connection tube having a valve. After the filling, remove the connection and immediately seal the 5 liter Tedlar™ bag by closing the valve.

Measure the concentration of a malodorous compound after a certain period of time using the standard detector system mentioned above. For the bag comprising hydrogen sulfide, measure the concentration of hydrogen sulfide after 24 hours, for the bag comprising ammonia, measure the concentration of ammonia after 3 hours. Repeat the test three times and take an average of these tests as the deodorant performance.

### EXAMPLES

Examples of the invention are set forth hereinafter by way of illustration and are not intended to be in any way limiting of the invention. The examples are not to be construed as limitations of the present invention since many variations thereof are possible without departing from its spirit and scope.

### Examples 1-3

Prepare 5 grams of a crosslinked sodium salt of polyacrylic acid gelling polymer having a preformed cuboid shape of 4 mm×4 mm×5 mm in a container. Premix ingredients described in Table 1 by stirring and homogenizing with a high-shear mixer to obtain a premix. Add the premix into the container comprising the gelling polymer and allow the gelling polymer to absorb the premix by keeping the mixture overnight. A deodorant composition having a cuboid shape and a surface area of 600 mm² to 680 mm² is then obtained. The deodorant composition has a light blue color.

TABLE 1
unit: grams

| | Example 1 | Example 2 | Example 3 |
|---|---|---|---|
| Copolymer of acrylic acid | 5.0 | 5.0 | 5.0 |
| and diethylamino ethyl | | | |
| methacrylate | | | |
| copper chloride (CuCl₂) | 0.025 | 0.037 | 0.065 |
| perfume | 2.0 | 2.0 | 2.0 |
| phenoxyethanol | 1.0 | 1.0 | 1.0 |
| SEESORB ™ | 0.2 | 0.2 | 0.2 |

| | |
|---|---|
| water | Balance to make the total |
| | of each example to be 95 |

Deodorant performance of the deodorant composition prepared in Examples 1-3 is measured by the test method described hereinabove. The initial concentration of hydrogen sulfide is 100 ppm and concentration of the hydrogen sulfide after 24 hours is measured and recorded in the below Table 2. The initial concentration of ammonia is 200 ppm and the concentration of ammonia after 3 hours is measured and recorded in the below Table 3.

TABLE 2
Deodorant performance on hydrogen sulfide

| | Example 1 | Example 2 | Example 3 |
|---|---|---|---|
| Concentration after 24 hours | 72 | 61 | 31 |
| (ppm) | | | |

TABLE 3
Deodorant performance on ammonia

| | Example 1 | Example 2 | Example 3 |
|---|---|---|---|
| Concentration after 3 hours | 10 | 10 | 10 |
| (ppm) | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition comprising
(i) from about 0.005% to about 2% by weight of a metallic deodorizing agent selected from the group consisting of copper, zinc, silver, platinum, nickel, iron, cobalt, a copper compound, a zinc compound, a silver compound, a platinum compound, a nickel compound, an iron compound, a cobalt compound and a mixture thereof,
(ii) an carboxylic group-containing gelling polymer, and
(iii) a liquid media,
wherein the deodorant composition has a three-dimensional shape selected from the group consisting of a block shape, a sphere shape, an ellipsoid shape, a cone shape and a cylinder shape and wherein the three-dimensional shape has a surface area of from about 24 mm² to about 2400 mm².

2. The deodorant composition of claim 1, wherein said metallic deodorizing agent is selected from the group consisting of copper chloride, copper sulfate, copper carbonate, zinc chloride, zinc sulfate, silver and a mixture thereof.

3. The deodorant composition of claim 1, wherein said carboxylic group-containing gelling polymer is a partially neutralized cross-linked polyacrylic acid.

4. The deodorant composition of claim 1, wherein said liquid media is water.

5. The deodorant composition of claim 1, wherein said three-dimensional shape is a block shape selected from the group consisting of a cube, a cuboid, a parallelepiped and an oblique rectangular prism.

6. The deodorant composition of claim 1, further comprising a deodorant polymer comprising cationically dissociating groups and anionically dissociating groups.

7. The deodorant composition of claim 6, wherein the deodorant polymer is grafted on a substrate selected from the group consisting of an aldohexose, a glucose, a starch, a cellulose and a mixture thereof.

8. The deodorant composition of claim 1, wherein the deodorant composition comprises adjunct materials selected from the group consisting of a perfume, a dye, a stabilizer, a UV absorbent, a preservative and a mixture thereof.

9. A deodorant product comprising a plurality of individual bodies of the deodorant composition of claim 1 packed in a package.

10. A process for making the deodorant composition of claim 1, comprising the steps of:
A. preparing a gelling polymer having a preformed three-dimensional shape in a container;
B. premixing and homogenizing any other ingredients of the deodorant composition to form a premix;
C. adding the premix into the container comprising the gelling polymer and keeping the mixture overnight;
wherein the preformed three-dimensional shape of the gelling polymer is selected from a block shape, a sphere shape, an ellipsoid shape, a cone shape and a cylinder shape, and wherein the weight ratio of the gelling polymer to the premix is from about 1:200 to about 1:1.
